(19) United States
(12) Patent Application Publication
(10) Pub. No.: US 2021/0158917 A1
(43) Pub. Date: May 27, 2021

(54) SYSTEM AND METHOD FOR CARE PATH PERFORMANCE ANALYSIS AND OPTIMAL PROVIDER NETWORK FORMATION

(71) Applicant: BaseHealth, Inc., Sunnyvale, CA (US)

(72) Inventors: Prakash MENON, Cupertino, CA (US); Prasanna DESIKAN, Sunnyvale, CA (US); Hadi ZARKOOB, Sunnyvale, CA (US); Hossein FAKHRAI-RAD, Los Altos, CA (US)

(21) Appl. No.: 17/105,827

(22) Filed: Nov. 27, 2020

Related U.S. Application Data
(60) Provisional application No. 62/941,049, filed on Nov. 27, 2019.

(51) Int. Cl. G16H 10/60; G06Q 40/08; G16H 40/20

(52) U.S. Cl. CPC G16H 10/60; G06Q 30/0282; G16H 40/20; G06Q 40/08

(57) ABSTRACT

A method of obtaining a virtual provider cluster is provided. The method includes obtaining a medical effectiveness per member, obtaining a preliminary virtual provider cluster, for each preliminary virtual provider cluster, calculating a MedicalEffectiveness as: MedicalEffectiveness=mean of the MedicalEffectiveness per member for all members associated with the preliminary virtual cluster; for each preliminary virtual provider cluster, calculating a RiskAdjustedCostEfficiency as RiskAdjustedCostEfficiency=sum of all MemberExpectedClaimAmount/sum of all MemberAllowedClaimAmount; and obtaining a virtual provider cluster by updating each preliminary virtual provider cluster with its corresponding MedicalEffectiveness and corresponding RiskAdjustedCostEfficiency.

### CROSS-REFERENCE TO RELATED APPLICATION(S)

This Application claims the benefit of U.S. Provisional Application 62/941,049 filed Nov. 27, 2019 in the U.S. Patent and Trademark Office, the disclosure of which is incorporated herein by reference in its entirety.

### BACKGROUND

### 1. Field

Apparatuses and methods consistent with exemplary embodiments relate to systems and methods for obtaining a virtual provider cluster.

### 2. Description of the Related Art

Various Healthcare organizations have a need to assess and influence Healthcare Provider behavior in order to improve the efficiency and effectiveness of member care.

Historically assessments of provider behavior have been limited to measuring and comparing Healthcare Providers on an individual basis.

FIG. 1 illustrates a layered framework for network health analysis. FIG. 1 shows various healthcare entities involved in individual care episodes for members. These entities illustrate that there are potentially complex interactions between these entities in providing care to members. Network optimization involves finding and implementing the best opportunities for cost saving, utilization management, referral paths, and physician performance enhancement based on various care quality and cost-efficiency measures.

### SUMMARY

Exemplary embodiments may address at least the above problems and/or disadvantages and other disadvantages not described above. Also, exemplary embodiments are not required to overcome the disadvantages described above, and may not overcome any of the problems described above.

According to an aspect of an example embodiment, a method of obtaining a virtual provider cluster comprises: obtaining a claims file and a referral file wherein all claims in the referral file are in the claims file; obtaining a medical effectiveness per member, comprising: obtaining a clean claims file by removing, from the claims file: claims with dates outside a current year and previous four years, claims with invalid claims dates, claims that have been subsequently adjusted, claims that have ERR, TOTAL, or ACCUM statuses, claims with negative claim amounts, duplicate claims, and reversed claims; obtaining a clean eligible claims file by filtering the clean claims file to include only members with at least six months of eligibility for the current year and each of the previous four years; calculating, by using the clean eligible claims file, a Medical Effectiveness per member, where Medical Effectiveness per member=((CurrentYearRisk−BaseYearRisk)/(current year−BaseYear)−(CurrentYearRisk−PreviousYearRisk))/CurrentYearRisk, where BaseYear is an earliest year for which a member has a calculated RAF score, the BaseYearRisk is the RAF calculated for a member for the BaseYear, the CurrentYearRisk is the RAF calculated for a member for the current year, and the PreviousYearRisk is the RAF calculated for the member for the previous year; calculating, by using the clean eligible claims file, a PopulationRafMultiplier as PopulationRafMultiplier=ClaimAllowedAmount/ExpectedTotalClaims, where ClaimAllowedAmount is a sum of all claim allowed amounts for the current year for all members and ExpectedTotalClaims is a sum of all expected total claims for all members; calculating, for each current year claim a MemberExpectedClaimAmount as MemberExpectedClaimAmount=ClaimAllowedAmount/PopulationRafMultiplier; obtaining a preliminary virtual provider cluster by: building, by using the referrals file, a plurality of graphs of referrals by following a path of referrer provider to referred provider until a node with no referred provider is reached; for each of the plurality of graphs, store one or more claims associated with each edge of the graph; merge all ones of the plurality of graphs that have a same provider node; define a preliminary virtual provider cluster as any merged graph having greater than four unique members associated with the one or more claims associated with each edge of the graph; for each preliminary virtual provider cluster, calculating a MedicalEffectiveness as: MedicalEffectiveness=mean of the MedicalEffectiveness per member for all members associated with the preliminary virtual cluster; for each preliminary virtual provider cluster, calculating a RiskAdjustedCostEfficiency as RiskAdjustedCostEfficiency=sum of all MemberExpectedClaimAmount/sum of all MemberAllowedClaimAmount; obtaining a virtual provider cluster by updating each preliminary virtual provider cluster with its corresponding MedicalEffectiveness and corresponding RiskAdjustedCostEfficiency.

According to an aspect of another example embodiment, a non-transitory computer-readable medium is provided, the non-transitory computer-readable medium storing thereon instructions which, when executed by a processor cause the processor to perform a method of obtaining a virtual provider cluster, the method comprising: obtaining a claims file and a referral file wherein all claims in the referral file are in the claims file; obtaining a medical effectiveness per member, comprising: obtaining a clean claims file by removing, from the claims file: claims with dates outside a current year and previous four years, claims with invalid claims dates, claims that have been subsequently adjusted, claims that have ERR, TOTAL, or ACCUM statuses, claims with negative claim amounts, duplicate claims, and reversed claims; obtaining a clean eligible claims file by filtering the clean claims file to include only members with at least six months of eligibility for the current year and each of the previous four years; calculating, by using the clean eligible claims file, a Medical Effectiveness per member, where Medical Effectiveness per member=((CurrentYearRisk-BaseYearRisk)/(current year−BaseYear)−(CurrentYearRisk−PreviousYearRisk))/CurrentYearRisk, where BaseYear is an earliest year for which a member has a calculated RAF score, the BaseYearRisk is the RAF calculated for a member for the BaseYear, the CurrentYearRisk is the RAF calculated for a member for the current year, and the PreviousYearRisk is the RAF calculated for the member for the previous year; calculating, by using the clean eligible claims file, a PopulationRafMultiplier as PopulationRafMultiplier=ClaimAllowedAmount/ExpectedTotalClaims, where ClaimAllowedAmount is a sum of all claim allowed amounts for the current year for all members and ExpectedTotalClaims is a sum of all expected total claims for all members; calculating, for each current year claim a MemberExpectedClaimAmount as MemberExpectedClaimAmount=ClaimAllowedAmount/PopulationRafMultiplier; obtaining a preliminary virtual provider cluster by: building, by using the referrals file, a plurality of graphs of referrals by following a path of referrer provider to referred provider until a node with no referred provider is reached; for each of the plurality of graphs, store one or more claims associated with each edge of the graph; merge all ones of the plurality of graphs that have a same provider node; define a preliminary virtual provider cluster as any merged graph having greater than four unique members associated with the one or more claims associated with each edge of the graph; for each preliminary virtual provider cluster, calculating a MedicalEffectiveness as: MedicalEffectiveness=mean of the MedicalEffectiveness per member for all members associated with the preliminary virtual cluster; for each preliminary virtual provider cluster, calculating a RiskAdjustedCostEfficiency as RiskAdjustedCostEfficiency=sum of all MemberExpectedClaimAmount/sum of all MemberAllowedClaimAmount; obtaining a virtual provider cluster by updating each preliminary virtual provider cluster with its corresponding MedicalEffectiveness and corresponding RiskAdjustedCostEfficiency.

### BRIEF DESCRIPTION OF THE DRAWINGS

The above and/or other aspects will become apparent and more readily appreciated from the following description of example embodiments, taken in conjunction with the accompanying drawings in which:

FIG. 1 is a diagram of a layered framework for network health analysis;

FIG. 2 is a diagram of entities involved in a care path and relationships thereamong;

FIG. 3 is a diagram of extraction of a virtual provider cluster;

FIG. 4 is a graph of each virtual provider cluster in a network, according to an example embodiment;

FIG. 5 is another graph of virtual provider clusters, according to an example embodiment;

FIG. 6 is a diagram of VPC 1 which is very efficient with a high rank of (2) and VPC 2 which has a low rank of (999) based on cost efficiency and clinical effectiveness; and

FIGS. 7 through 14 each illustrate a portion of a flow chart of a method according to an example embodiment.

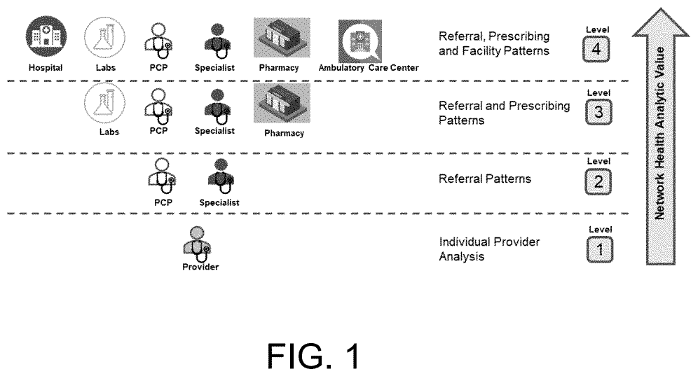

### DETAILED DESCRIPTION

Reference will now be made in detail to example embodiments which are illustrated in the accompanying drawings, wherein like reference numerals refer to like elements throughout. In this regard, the example embodiments may have different forms and may not be construed as being limited to the descriptions set forth herein.

It will be understood that the terms “include,” “including”, “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names—this document does not intend to distinguish between components that differ in name but not function.

Matters of these example embodiments that are obvious to those of ordinary skill in the technical field to which these exemplary embodiments pertain may not be described here in detail.

Example embodiments may address at least the above problems and/or disadvantages and other disadvantages not described above. Also, example embodiments are not required to overcome the disadvantages described above, and may not overcome any of the problems described above.

One or more example embodiments may enable the measurement of provider behavior in the context of individual member care episodes. Measurements in the context of member care episodes, may allow a comprehensive understanding of provider behavior within the often-complex referral paths that the member traverses during any given care episode. Measurement in the context helps in identification of over and under referrals and individuals who have a disproportionate impact on the overall performance of a care path.

One or more example embodiments may enable the measurement of provider behavior in the context of specific medical specialties. These measurements may allow for the formation of provider networks that are optimal for treatment of specific conditions.

One or more example embodiments may enable the comparison of provider behavior normalized over externalities such as incoming patient health, social background, ethnicities etc. By normalizing over externalities, the comparisons of results may become equitable and explainable. An objective of the measurement is to modify provider behavior, and the fairness of the measurement may be important.

One or more example embodiments are related to identifying individual care paths for members through referral patterns across different entities. These individual care paths are then compared and combined to build optimal networks of providers, labs, specialty clinics, hospitals and other facilities that enable effective and efficient care delivery both at an individual level and at an aggregated population level. The framework may provide a way to model the interactions in a care path of the member. Subject to the availability of the data, different levels of interactions can be used to define a network of care paths for the members.

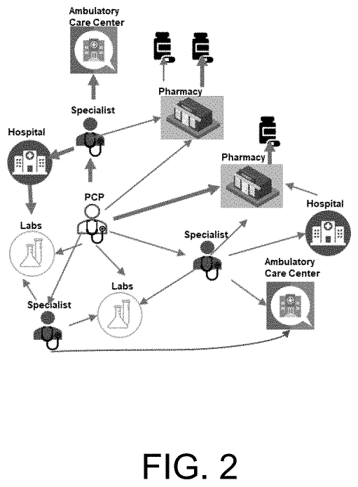

FIG. 2 is a diagram of entities involved in a care path and relationships thereamong. FIG. 2 illustrates how the different entities and referrals can be captured in an entity relationship graph, thus enabling the modeling of care paths.

As a first step to model the care paths, the underlying elements and their relationships are defined and captured. These include:

Defining and identifying participating entities: Deciding the key entities that would participate in the network and defining a methodology to identify them. This is based on the hypothesis being tested. E.g. If the hypothesis being tested concerns the use of testing by primary care providers, the scope would only need to include members, PCPs, and Laboratories.

Defining and identifying relationships between entities: The framework provides a way to model the interactions in a care path of the member. As mentioned above, one or more example embodiments may model interactions within care paths, the relationships between entities are also identified based on participation in care paths. Thus, there could be multiple relationships between identical entities that contribute to different overall care paths. The primary basis for inferring relationships is member referral data.

Define “weight” metric for entities: Identified entities are weighed based on different criteria. For example, while modeling facilities, they could be weighed by their size—such as the number of beds available, or presence or absence of a preferred provider relationship. The weights are used to include non-optimizable entity level constraints when constructing optimal provider networks.

Define “distance” for relationships: Identified relationships are weighted using a distance measure. For example, the actual geographical distance between providers could be a defined distance measure. Distances are used to include non-optimizable relationship level constraints when constructing optimal provider networks.

One or more example embodiments may identify measures of interest (performance measures) that are related to functional/business metrics relevant to the hypothesis being tested. For example, clinical effectiveness, risk-adjusted cost efficiency are measures of interest when comparing providers or groups of providers.

One or more example embodiments may analyze the care paths extracted and rank them using the measures of interest. Individual providers can also be ranked based on their participation in high performing care paths.

One or more example embodiments may use network analysis and link analysis techniques to identify providers that are essential to the referral communications in the network.

One or more example embodiments may use all of the above information to build the optimal provider network.

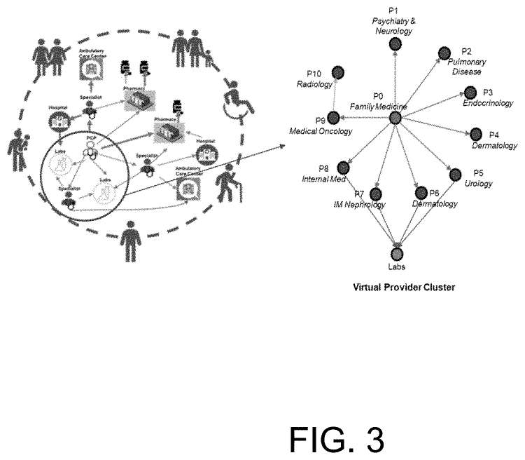

An example embodiment of extracting Virtual Provider Clusters (VPCs): For a given population of members, we obtain a year's worth of claims. In addition, we obtain referral data for each episode of care for the member. For each member in the population, we extract the care path for each episode of care, by following the referrals within the episode of care. Note that the member will participate in multiple care paths, (for different episodes of care), as will providers. We then identify care paths that have been traversed by minimum threshold of members (e.g. five unique members). Each such care path is designated a Virtual Provider Cluster (VPC). Additional conditions can be placed on the care path extraction (e.g. the primary diagnosis for the members that flow through the care path). The VPC thus extracted is by definition an implicit sub network within the larger provider network, that has been traversed by multiple members. FIG. 3 illustrates a single VPC extracted from member data. Multiple members have entered this network through family medicine provider P0, who referred them to specialists P1, P2, P3, P4, P5, P6, P7, P8, and P9. Specialist P9 further referred the members to P10. Also, multiple specialists P5, P6, P7, P8 referred the members to a laboratory for lab tests.

Determining Performance Measures: This example implementation uses two performance measures, clinical effectiveness and risk-adjusted cost efficiency. The performance measures are determined based on the following methodology.

Risk Adjusted Cost Efficiency: Assuming current year is “t” we use claims from previous year “t−1” to calculate the RAF score for each member. Using the calculated RAF score and base rate published by CMS we arrive at an expected total claims value for each member. In a population of n members.

$$PopulationRafMultiplier = \frac{[ {\sum }_{k = 1}^{n} ClaimAllowedAmount ]}{[ {\sum }_{k = 1}^{n} ExpectedTotalClaims ]}$$

$${MemberExpectedClaimAmount}_{i} = \frac{{ClaimAllowedAmount}_{i}}{{PopulationRafMultipliter}_{i}}$$

$$Where i is an individual claim$$

$${RiskAdjustedCostEfficiency}_{v} = \frac{\sum {MemberExpectedClaimAmount}_{i}}{\sum {MemberAllowedClaimAmount}_{i}}$$

where i ranges over all claims associated with the vpc v

This is measured as the difference in slope of the risk change for the current year with the slope of the risk change for all available data for the member. The risk can be measured with any risk methodology, for the present embodiment, it is calculated using RAF methodology from CMS.

$$MedicalEffectiveness = \frac{\begin{matrix}\{ [ \frac{( CurrentYearRisk - BaseYearRisk )}{CurrentYear - BaseYear} ] - \\ [ CurrentYearRisk - PreviousYearRisk ] \}\end{matrix}}{CurrentYearRisk}$$

While these measures are well defined and applicable for Medicare Advantage population, the framework for defining these measures allows flexibility in modifying and expanding such measures to include a wide range of both clinical and cost measures that apply to other business use cases.

Ranking VPCs: Once the clinical effectiveness and cost efficiency scores (or any such measures) are computed, we determine statistical thresholds (that can be complemented or combined with measures defined by domain experts) to identify high-performing efficient VPCs based on these measures. These measures can then be used to determine the scores and thresholds for stratifying VPCs. Such stratification may help answer questions of relevance such as:
How would these clusters deliver higher quality care?
How would these clusters deliver cost savings?

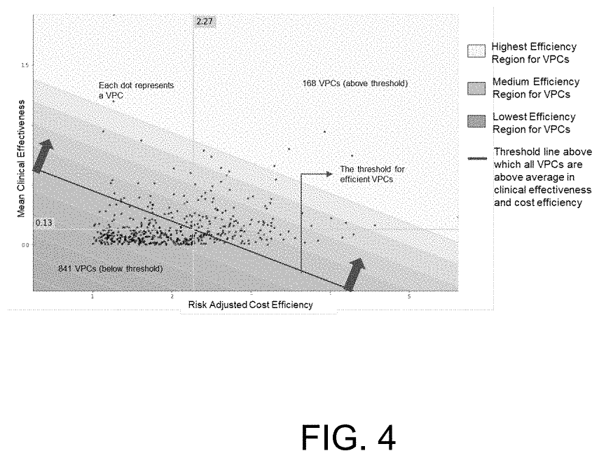

The method to stratify the VPCs based on the combination of the different measures may vary based on the problem being addressed. We describe an example approach to stratify VPCs in FIG. 4 below. FIG. 4 is a graph of each virtual provider in a network, according to an example embodiment. The oblique areas represent virtual private clusters that are comparable in their combined mean clinical effectiveness and risk adjusted cost efficiency. Virtual provider clusters in the right, top quadrant are better performers than clusters in the bottom, left quadrant. The score of a VPC in this approach is given by weighted function of clinical effectiveness and cost efficiency, as follows:

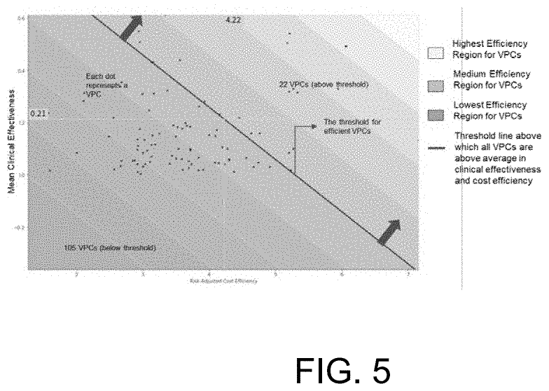

VPC score=w₁×Clinical Effectiveness+w₂×Cost Efficiency

where the parameters w₁ and w₂ adjust the contribution of the studied performance measures to the overall score. All VPCs that score above a certain statistically defined threshold will perform overall better than the entire network put together in terms of both cost efficiency and clinical effectiveness (FIG. 4). These are the highly efficient VPCs, that bring in both high-quality care and deliver cost savings. Such analysis can also be conducted for disease-specific networks, such as type 2 diabetes shown in the FIG. 5. FIG. 5 is a graph similar to FIG. 4, but limited to claims that are related to a specialty, in this case, type 2 diabetes, according to an example embodiment. The virtual provider clusters in the top, right quadrant are better performers in treating type 2 diabetes than clusters in the bottom, left quadrant.

The approach allows us to stack rank VPCs by combined performance measures. Thus, using statistically robust techniques, an optimal provider network is built by choosing the VPCs that are both clinically effective and cost efficient after adjusting for risk.

Determining Provider Performance: Provider ranks are computed based on their participation in highly performant VPCs. The provider ranks are thus highly applicable to any population with similar prevalence of conditions. The provider score in the exemplary approach is given by:

$$ProviderRank = \frac{{\sum }_{i = 1}^{n} {e}^{- {Rank}_{i}}}{n}$$

where the provider participates in n VPCs, and Rank is the rank of the ith VPC, by VPC score.

Determining Critical Providers for the network: Essentially what these measures reveal is the set of providers who are critical to the network because of their influence and cannot be judged solely by their clinical effectiveness and cost efficiency. In an example approach, social network analysis measures of hub centrality (providers that are sending referrals to a wide range of others each of whom has many others referring to them), authority (providers that are being referred to from a wide range of others each of whom sends referrals to a large number of others), and contribution (providers that bridge different sub networks that don't normally out refer) were used to identify node criticality.

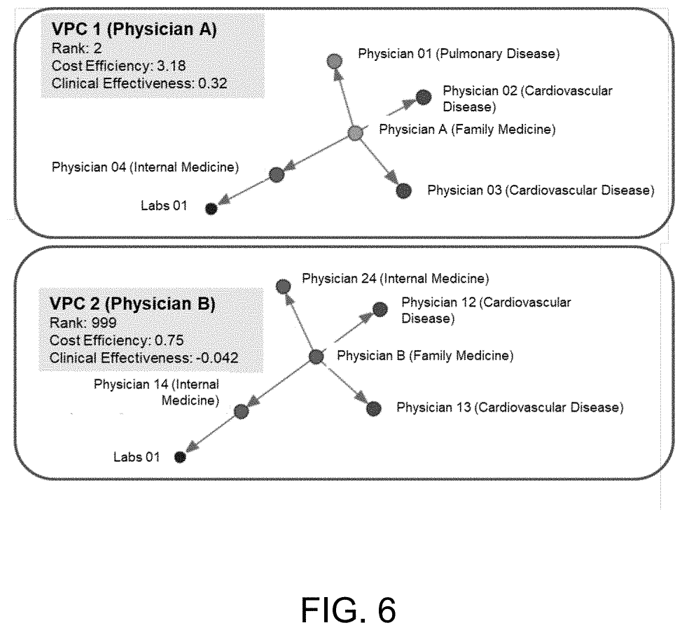

Build the Optimal Provider Network: The construction of VPCs allows comparison among them based on performance measures such as the clinical effectiveness and cost efficiencies. FIG. 6 illustrates two VPCs who have similar members and referral patterns. However, VPC 1 is very efficient with high rank (2), whereas VPC 2 is ranked low (999). Based on their cost spent and the efficiency, it is determined that if the members that are treated by VPC 2 were treated by VPC 1 or if the providers in VPC 2 achieved similar efficiency as VPC 1, the analyzed cost savings as an example would be, $1,021.38 per member, with better clinical effectiveness. This information is useful while building the optimal provider network.

Once we determine the ranked list of primary care providers, the ranked list of specialty providers and the list of critical providers that need to be present in the network, we build the optimal provider network as follows:

Identify the specialty that the network will address, if the network is not specialty oriented, we use the overall ranks of the VPCs otherwise we use the specialty specific ranks.

Start with the top ranked VPCs, if the network identified fulfils the resourcing needs for the population at hand we are done.

If additional providers are required, we examine the next ranked VPC. If the next ranked VPC is below efficient VPC threshold we switch to looking at individual providers. If the VPC is above efficient VPC threshold we merge the add the new VPC to the network. When picking the additional VPC we prioritize the VPCs that have providers with high network criticality with respect to the currently formed network. If the updated network fulfils the resourcing needs for the population at hand we are done otherwise repeat this step. If we hit below efficient threshold VPCs go to next step.

From the ranked list of providers remove providers that have already been included in the currently formed network. Order the remaining providers by rank and criticality. Add individual providers to the formed network until resourcing needs are met.

This methodology is flexible and can be extended to include new constraints based on physician bandwidth, network adequacy, network access, etc.

A network intelligence platform, in addition to the aforementioned capabilities, may deliver expansion of the analytics based on custom defined measures, incorporating policy and other constraints, and is applicable to other business cases such as opioid utilization or disease management where provider referral networks influence the business problem.

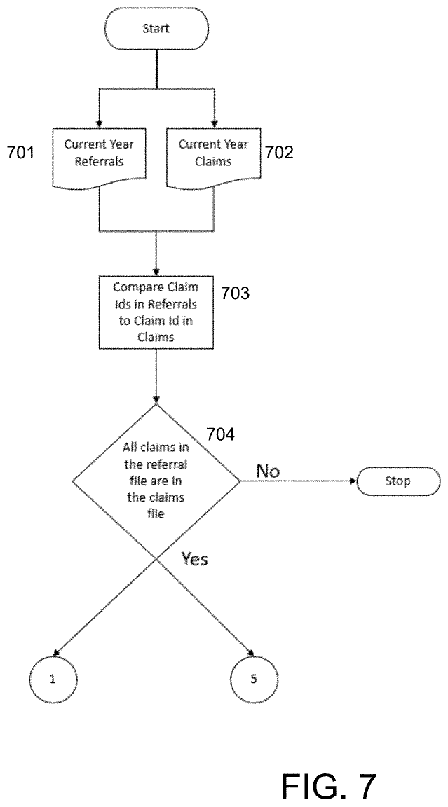

FIGS. 7 through 14 each illustrate a portion of a flow chart of a method according to an example embodiment.

A claims file is an industry standard claims file and normally has the following information:

MEMBER_SK—member id

CLMS_ID—claim id

CLMS_LINE—claim line number

CLM_THRU_DATE_SK—date of claim

CLAIM_ADJTO—claim id that this claim is an adjustment to

CLAIM_STATUS—status of the claim

CLAIM_AMT—amount of the claim

CLAIM_ALLOWED_AMT—allowed amount of the claim

PAID_INS_AMT—insurance paid amount on the claim

PAID_COINS_AMT—coinsurance amount paid on the claim

PAID_COPAY_AMT—copay amount on the claim

PAID_DEDUCT_AMT—deductible amount on the claim

PAID_NONDEDUCT_AMT—non-deductible amount on the claim

PAID_WITHHELD_AMT—withheld amount on the claim

PRIMARY_DIAG—Primary diagnosis code for the claim

DIAG2—Additional diagnosis code for the claim

DIAG3—Additional diagnosis code for the claim

DIAG4—Additional diagnosis code for the claim

DIAG5—Additional diagnosis code for the claim

PROCEDURE_CPT—Primary Procedure code for the claim

PROCEDURE_DSCR—Procedure description for the claim.

A referral file contains information about how the member was referred through different providers and the claim associated with the referral, and it contains the following:

MEMBER_SK—member id

CLMS_ID—claim id associated with this referral

REFERRING_NETWORK_NPI—the National provider identifier for the referring provider

REFERRING_NETWORK_NAME—Name of the referring provider

NETWORK_NPI—National provider identifier for the referred provider

NETWORK_NAME—Name of the referred provider

NETWORK_NETWORK—Provider network the Referred provider belongs to

PROVIDER_TIN—Referred provider tax id number

NETWORK_SPECIALTY—Referred provider Specialty

PROVIDER_OFFICE NAME—Referred provider office name

PROVIDER_ZIP—Referred provider zip.

An eligibility file contains information about the coverage eligibility of the member, and it has the following information:

MEMBER_SK—member id

ELIGIBLE—whether member is eligible for coverage

YEARMONTH—year and month the eligible flag refers to.

A diagnosis file is provided because some claims may not have primary diagnosis associated with them. It contains the following information:

MEMBER SK—member id

CLMS_ID—claim id

DIAG_SEQUENCE—order of the diagnosis

DIAG_CD—ICD code for the diagnosis

ICD10_IND—denotes if the DIAG_CD is an ICD10 code or ICD9 code.

As shown, starting at FIG. 7, a method according to an example embodiment begins by receiving current year referrals 701 and current year claims 702. The claim IDs in the referrals are compared to the claim IDs in the claims at 703. At 704, it is determined whether all the claims in the referral file are in the claims file. If the answer is no, the operations stop. If the answer is yes, the operations proceed to \textcircled{1} and \textcircled{5}.

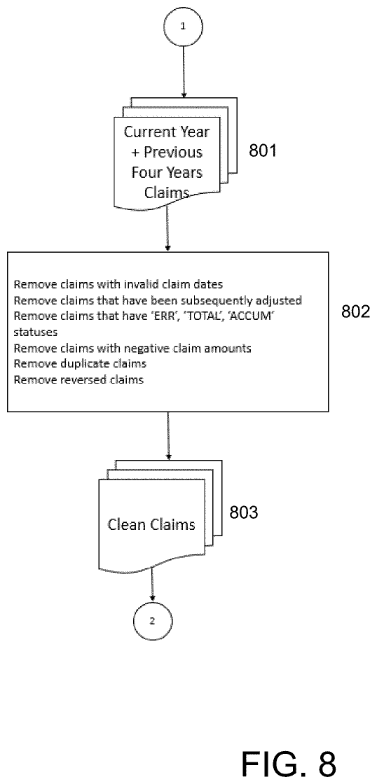

As shown in FIG. 8, the claims from the current year and previous 4 years are received at 801. At 802, the claims with invalid claim dates, the claims that have been subsequently adjusted, the claims that have ERR, TOTAL, or ACCUM statuses, the claims with negative claim amounts, duplicate claims, and reversed claims are removed. Thus, the clean claims are obtained at 803, and the operations proceed to \textcircled{2}.

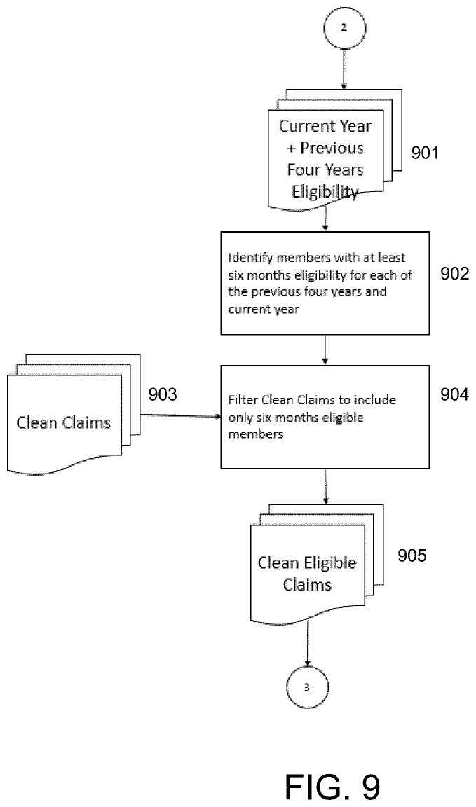

As shown in FIG. 9, the claims from the current year and previous 4 years of eligibility are received at 901. At 902, members with at least six months eligibility are identified for each of the current year and the previous four years. At 903, the clean claims are received, and at 904, the clean claims are filtered to include only at least six months eligible members. Thus, the clean, eligible claims are obtained at 905, and the operations proceed to \textcircled{3}.

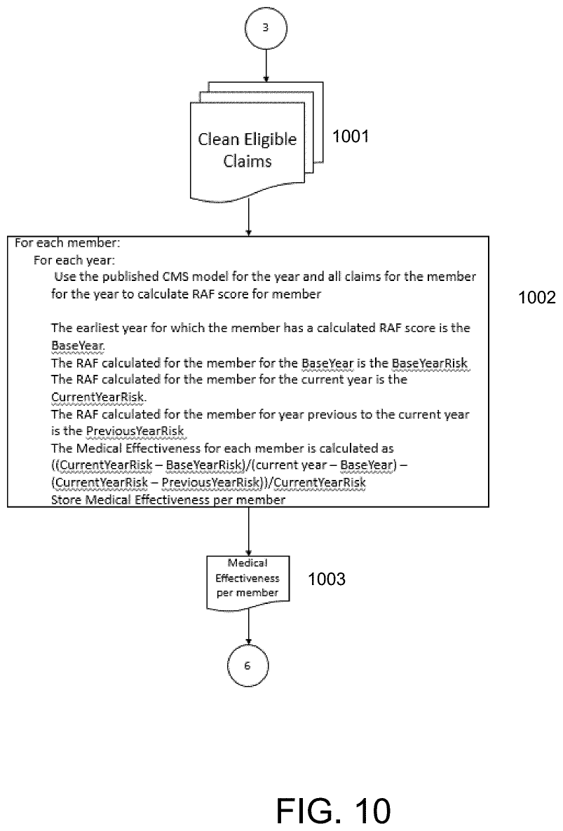

As shown in FIG. 10, the clean eligible claims are received at 1001. At 1002, for each member, for each year, the published CMS model is used for the year and all claims for the member for the year are used to calculate the RAF score for each member. The earliest year for which the member has a calculated RAF score is the BaseYear. The RAF calculated for the member for the Base Year is the Base YearRisk. The RAF calculated for the member for the current year is the CurrentYearRisk. The RAF calculated for the member for year previous to the current year is the PreviousYearRisk. The Medical Effectiveness for each member is calculated as ((CurrentYearRisk−BaseYearRisk)/(current year−BaseYear)−(CurrentYearRisk−PreviousYearRisk))/CurrentYearRisk. The Medical Effectiveness per member is stored. Thus, at 1003, the Medical Effectiveness per member is obtained, and the operations proceed to \textcircled{6}.

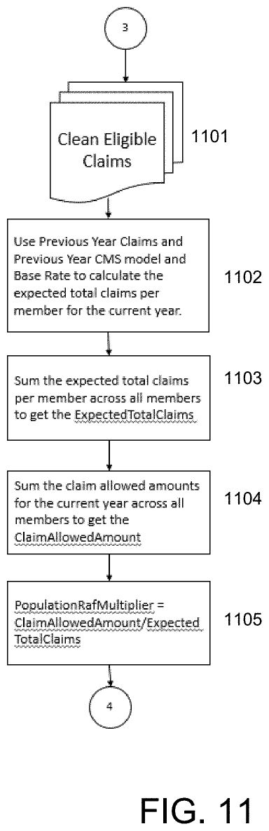

As shown in FIG. 11, the clean eligible claims are obtained at 1101. At 1102, the previous year claims and the previous year CMS model and the Base Rate are used to calculate the expected total claims per member for the current year. At 1103, the expected total claims per member across all members is summed to get the ExpectedTotalClaims. At 1104, the claim allowed amounts are summed for the current year across all the members to get the ClaimAllowedAmount. At 1105, the PopulationRafMultiplier is calculated as: PopulationRafMultiplier=ClaimAllowedAmount/ExpectedTotalClaims, and the operations proceed to \textcircled{4}.

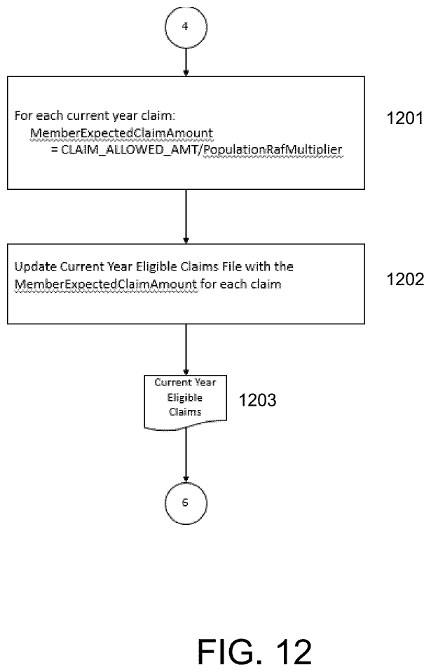

As shown in FIG. 12, at 1201, for each current year claim, the MemberExpectedClaimAmount is calculated as: MemberExpectedClaimAmount=CLAIM_ALLOWED_AMT/PopulationRafMultiplier. At 1202 The Current Year Eligible Claims file is updated with the MemberExpectedClaimAmount for each claim. Thus, The Current Year Eligible Claims are obtained at 1203, and the operations proceed to \textcircled{6}.

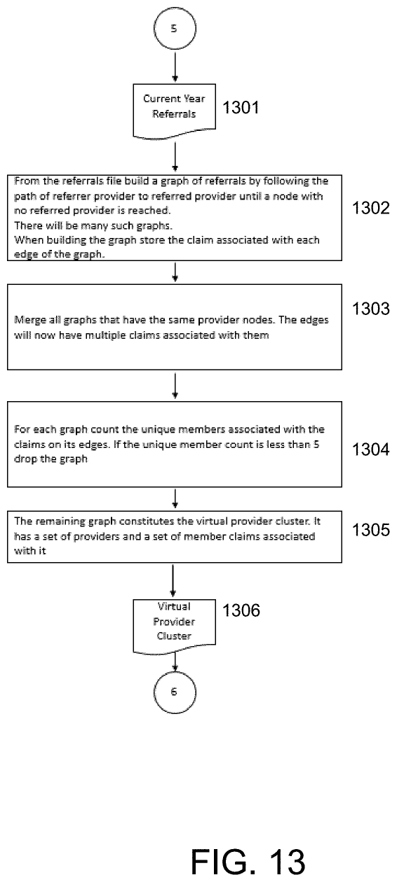

As shown in FIG. 13, at 1301, the Current Year Referrals are received. At 1302, From the referrals file, a graph of referrals is built by following the path of referrer provider to referred provider until a node with no referred provider is reached. Many graphs are generated. When building the graph, the claim associated with each edge of the graph is stored. At 1303, All graphs that have the same provider nodes are merged. the edges will now have multiple claims associated with them. At 1304, for each graph, the unique members associated with the claim on its edges are counted. If the unique member count is less than 5, the graph is dropped. At 1305, the remaining graph constitutes the virtual provider cluster. It has a set of providers and a set of member claims associated with it. Thus, at 1306, the virtual provider cluster is obtained, and the operations proceed to \textcircled{6}.

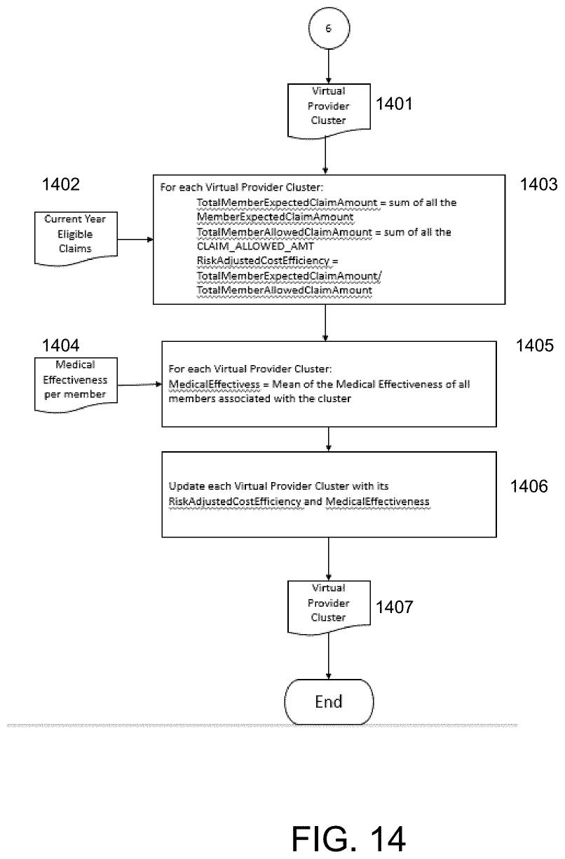

As shown in FIG. 14, the virtual provider cluster is received at 1401. The current year eligible claims are received at 1402. At 1403, for each Virtual Provider Cluster, the TotalMemberExpectedClaimAmount is calculated as: TotalMemberExpectedClaimAmount=sum of all the MemberExpectedClaimAmount. The TotalMemberAllowedClaimAmount is calculated as: TotalMemberAllowedClaimAmount=sum of all the CLAIM_ALLOWED_AMT. The RiskAdjustedCostEfficiency is calculated as: RiskAdjustedCostEfficiency=TotalMemberExpectedClaimAmount/TotalMemberAllowedClaimAmount. At 1404, the Member effectiveness per member is received. At 1405, for each virtual provider cluster, the MedicalEffectiveness is calculated as MedicalEffectiveness=mean of the medical effectiveness of all members associated with the cluster. At 1406, each virtual provider cluster is updated with its RiskAdjustedCostEfficiency and MedicalEffectiveness. Thus, the virtual provider cluster is obtained at 1407, and the operations end.

The methods and operations described above with respect to example embodiments can be implemented, at least in part, in digital electronic circuitry, analog electronic circuitry, or in computer hardware, firmware, software, or a combination thereof. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or other device or on multiple device at one site or distributed across multiple sites and interconnected by a communication network. Also, functional programs, codes, and code segments for accomplishing features described herein can be easily developed by programmers skilled in the art. Method steps associated with the example embodiments can be performed by one or more programmable processors executing a computer program, code or instructions to perform functions (e.g., by operating on input data and/or generating an output). Method steps can also be performed by, and apparatuses described herein can be implemented as, special purpose logic circuitry, e.g., a field programmable gate array (FPGA) or an application-specific integrated circuit (ASIC), for example.

A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory (ROM) (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Computer-readable non-transitory media includes all types of computer readable media, including magnetic storage media, optical storage media, flash media and solid state storage media. It should be understood that software can be installed in and sold with a central processing unit (CPU) device. Alternately, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

It may be understood that the example embodiments described herein may be considered in a descriptive sense only and not for purposes of limitation. Descriptions of features or aspects within each exemplary embodiment may be considered as available for other similar features or aspects in other exemplary embodiments.

While example embodiments have been described with reference to the figures, it will be understood by those of ordinary skill in the art that various changes in form and details may be made therein without departing from the spirit and scope as defined by the following claims.

What is claimed is:

1. A method of obtaining a virtual provider cluster, the method comprising:
obtaining a claims file and a referral file wherein all claims in the referral file are in the claims file;
obtaining a medical effectiveness per member, comprising:
obtaining a clean claims file by removing, from the claims file: claims with dates outside a current year and previous four years, claims with invalid claims dates, claims that have been subsequently adjusted, claims that have ERR, TOTAL, or ACCUM statuses, claims with negative claim amounts, duplicate claims, and reversed claims;
obtaining a clean eligible claims file by filtering the clean claims file to include only members with six months of eligibility for the current year and the previous four years;
calculating, by using the clean eligible claims file, a Medical Effectiveness per member, where Medical Effectiveness per member=((CurrentYearRisk−BaseYearRisk)/(current year−BaseYear)−(CurrentYearRisk−PreviousYearRisk))/CurrentYearRisk, where BaseYear is an earliest year for which a member has a calculated RAF score, the BaseYearRisk is the RAF calculated for a member for the BaseYear, the CurrentYearRisk is the RAF calculated for a member for the current year, and the PreviousYearRisk is the RAF calculated for the member for the previous year;
calculating, by using the clean eligible claims file, a PopulationRafMultiplier as PopulationRafMultiplier=ClaimAllowedAmount/ExpectedTotalClaims, where ClaimAllowedAmount is a sum of all claim allowed amounts for the current year for all members and ExpectedTotalClaims is a sum of all expected total claims for all members;
calculating, for each current year claim a MemberExpectedClaimAmount as MemberExpectedClaimAmount=ClaimAllowedAmount/PopulationRafMultiplier;
obtaining a preliminary virtual provider cluster by:
building, by using the referrals file, a plurality of graphs of referrals by following a path of referrer provider to referred provider until a node with no referred provider is reached;
for each of the plurality of graphs, store one or more claims associated with each edge of the graph;
merge all ones of the plurality of graphs that have a same provider node;
define a preliminary virtual provider cluster as any merged graph having greater than four unique members associated with the one or more claims associated with each edge of the graph;
for each preliminary virtual provider cluster, calculating a MedicalEffectiveness as: MedicalEffectiveness=mean of the MedicalEffectiveness per member for all members associated with the preliminary virtual cluster;
for each preliminary virtual provider cluster, calculating a RiskAdjustedCostEfficiency as RiskAdjustedCostEfficiency=sum of all MemberExpectedClaimAmount/sum of all MemberAllowedClaimAmount; and
obtaining a virtual provider cluster by updating each preliminary virtual provider cluster with its corresponding MedicalEffectiveness and corresponding RiskAdjustedCostEfficiency.

2. A non-transitory computer-readable medium storing thereon instructions which, when executed by a processor cause the processor to perform a method of obtaining a virtual provider cluster, the method comprising:
obtaining a claims file and a referral file wherein all claims in the referral file are in the claims file;
obtaining a medical effectiveness per member, comprising:
obtaining a clean claims file by removing, from the claims file: claims with dates outside a current year and previous four years, claims with invalid claims dates, claims that have been subsequently adjusted, claims that have ERR, TOTAL, or ACCUM statuses, claims with negative claim amounts, duplicate claims, and reversed claims;
obtaining a clean eligible claims file by filtering the clean claims file to include only members with six months of eligibility for the current year and the previous four years;
calculating, by using the clean eligible claims file, a Medical Effectiveness per member, where Medical Effectiveness per member=((CurrentYearRisk−BaseYearRisk)/(current year−BaseYear)—(CurrentYearRisk−PreviousYearRisk))/CurrentYearRisk, where BaseYear is an earliest year for which a member has a calculated RAF score, the BaseYearRisk is the RAF calculated for a member for the BaseYear, the CurrentYearRisk is the RAF calculated for a member for the current year, and the PreviousYearRisk is the RAF calculated for the member for the previous year;
calculating, by using the clean eligible claims file, a PopulationRafMultiplier as PopulationRafMultiplier=ClaimAllowedAmount/ExpectedTotalClaims, where ClaimAllowedAmount is a sum of all claim allowed amounts for the current year for all members and ExpectedTotalClaims is a sum of all expected total claims for all members;
calculating, for each current year claim a MemberExpectedClaimAmount as MemberExpectedClaimAmount=ClaimAllowedAmount/PopulationRafMultiplier;
obtaining a preliminary virtual provider cluster by:
building, by using the referrals file, a plurality of graphs of referrals by following a path of referrer provider to referred provider until a node with no referred provider is reached;
for each of the plurality of graphs, store one or more claims associated with each edge of the graph;
merge all ones of the plurality of graphs that have a same provider node;
define a preliminary virtual provider cluster as any merged graph having greater than four unique members associated with the one or more claims associated with each edge of the graph;
for each preliminary virtual provider cluster, calculating a MedicalEffectiveness as:
MedicalEffectiveness=mean of the MedicalEffectiveness per member for all members associated with the preliminary virtual cluster;
for each preliminary virtual provider cluster, calculating a RiskAdjustedCostEfficiency as RiskAdjustedCostEfficiency=sum of all MemberExpectedClaimAmount/sum of all MemberAllowedClaimAmount; and
obtaining a virtual provider cluster by updating each preliminary virtual provider cluster with its corresponding MedicalEffectiveness and corresponding RiskAdjustedCostEfficiency.

3. A method of ranking virtual provider clusters, the method comprising:
for each of a plurality of virtual provider clusters (VPC), determining a VPC score as:
VPC score=w₁×Clinical Effectiveness+w₂×Cost Efficiency
where the parameters w₁ and w₂ adjust a contribution of studied performance measures to the overall score, and
ranking the plurality of VPC according to the determined VPS scores.
